(19) United States
(12) Patent Application Publication
(10) Pub. No.: US 2005/0034183 A1
(43) Pub. Date: Feb. 10, 2005

(54) Non-human animal model for analysis of the original and therapy of organ fibrosis

(76) Inventors: Rolf Gebhardt, Leipzig (DE); Hermann Bujard, Heidelberg (DE)

Correspondence Address: EDWARDS & ANGELL, LLP, P.O. BOX 55874, BOSTON, MA, 02205, US

(21) Appl. No.: 10/490,885

(22) PCT Filed: Sep. 27, 2002
(86) PCT No.: PCT/EP02/10908

(30) Foreign Application Priority Data
Sep. 28, 2001 (DE) 101 48 195.0

(51) Int. Cl. A01K 67/027; C12N 5/06

(52) U.S. Cl. 800/18; 435/354

(57) ABSTRACT

The present invention provides a nonhuman animal model for investigating the cause of and the therapy for organ fibrosis. Furthermore, the invention is directed to an animal model comprising a double transgenic, nonhuman animal having the ability to develop a fibrotic phenotype in a conditional and organ-specific manner. Moreover, the invention provides a method for generating the double transgene nonhuman animal.

The present invention is directed to a nonhuman animal model for investigating the cause of and therapies for organ fibrosis. The animal model described herein comprises a double transgenic, nonhuman animal having the ability to develop fibrotic phenotypes in a conditional and organ-specific manner.

### BACKGROUND OF THE INVENTION

The term “fibrosis”, also frequently known as “sclerosis”, refers to a pathological increase in the proliferation of connective tissue, whereby one or more body organs accumulate large quantities of extracellular matrix and connective tissue-like material. This localized increase of connective tissue material often leads to significant damage to those cells located in the surrounding tissue. As a response to this fibrotic effect, the surrounding tissue often undergoes a scarring type reaction.

Depending on the severity of the scarring reaction of the tissue as described above, the fibrosis may be reversible, in whole or in part. Often, however, fibrotic tissue will develop into a clinically significant lesion, which can lead to organ failure or even death of the affected patient. This outcome is especially likely in patients having other secondary conditions which may otherwise favor the formation of fibrotic lesions, e.g. alcohol abuse, intoxication, viral infections (such as hepatitis).

Liver cirrhosis, a final clinical stage for many patients having fibrosis associated with liver disease, is a leading cause of death worldwide. In the USA, liver cirrhosis represents the fifth leading cause of death for persons less than 65 years of age. The appreciated annual medical costs for patients suffering from liver cirrhosis are estimated to be between 6 to 8 trillion dollars in the USA alone.

Many of the factors that play a role in the formation of fibrotic lesions are known. However, there are presently no effective pharmaceutical compounds nor clinical strategies that can prevent the process of the fibrotic lesions, or even hinder the development of such lesions.

Therefore, the present situation with respect to known therapies and treatments for fibrosis is far from satisfactory and will likely result in a higher incidence of patient mortality. Moreover, little is known about the detail over the variety of mechanisms which participate in the organ regenerative process following a fibrotic lesion.

Medical therapies directed to preventing fibrosis do not represent the means of choice for stimulating organ regeneration per se. Because fibrosis patients typically begin their treatment by searching for and consulting with a physician after the fibrosis has already developed, the chances of successfully treating these lesions is slightly less than in the case of other diseases.

Presently, animal models are used for investigating liver fibrosis and anti-fibrosis agents comprising carbon tetrachloride induced liver damage (McLean E. K. et al., Br J Exp Pathol 1969; 50:502-506), and ligation or obstruction of the bile duct (Gerling B. et al., J. Hepatol. 1996; 25:79-84). In the first case, the fibrosis is released through simultaneous destruction of hepatocytes (in the pericentral region). In the second case, the fibrosis develops due to the continuous injury of hepatocytes as a result of a significant increase in the concentration of acids released from the gall bladder.

Known animal models used to study fibrosis have many disadvantages because of the difficulty in monitoring the progress of fibrotic disease (i.e. high incidence of early mortality) and the differences in the timing of disease progression and in the methodology used to measure fibrotic injury in an affected organ (Gebhardt R. and Reichen J., J. Hepatology 1994, 20:684-691).

Other transgenic animal models have been established, which have been useful in characterizing TGF-β as one of the most important cytokines that both induces and supports the process of fibrogenesis.

However, as noted above, these transgenic models have numerous disadvantages. One model has described, for example, for the expression of various cytokines under the control of an albumin promoter (Sanderson N. et al., PNAS USA 1995, 92:2572-2576) leading to early and continuous TGF-β production. But, due to the early emergence of a fatal nephropathy, the transgenic animals in this paradigm had relatively limited lifespans (Bisgaard H. C. and Thorgeirsson S., Clin Lab Med 1996, 16:325-33).

Another known transgenic model is based upon a lipo-polysaccharide inducible CRP promoter. However, the emergence of a continuous fibrotic process was missing because the animals quickly developed a tolerance to the LPS (Kanzler S. et al., Am J Physiol 1999, 276:61059-61068). Moreover, a further disadvantage of this system is that the LPS itself acts as a pathogen with respect to the liver (Heller J. et al., J Hepatol 2000; 33:376-381; Hiraoka E. et al., Liver 1995; 15:35-38) and further interferes with the fibrotic process in an unfavorable manner.

Based on the above-described disadvantages of transgenic and non-transgenic animal models known in the state of the art, such models are insufficiently suited to examine the causal mechanisms of fibrosis and potential therapies for fibrosis.

Therefore, there exists a need for a transgenic animal model, which provides for the controlled induction of fibrosis through the use of a cytokine, without interfering or otherwise damaging the organism.

Furthermore, there exists a need for a transgenic animal model, which provides for characterizing the mechanism of fibrosis development in an organ of interest.

These problems are solved through the claims of the present invention.

### SUMMARY OF THE INVENTION

The present invention provides for a nonhuman animal model for organ fibrosis, whereby this model comprises a double transgenic, nonhuman animal.

In one embodiment, the invention concerns a transgenic, nonhuman animal, which comprises a first recombinant gene that is stably integrated into the genome, said gene coding for a cytokine, whereby the cytokine is expressed in a conditional and organ-specific manner and whereby this expression leads to organ fibrosis.

The term “conditional” is understood to refer to a dependence of some activity on a stimulus or signal. The term “conditional expression” is understood to refer to the dependence of some kind of expression on a stimulus or other signal. The preferred stimulus according to the present invention is doxycycline.

“Organ-specific expression” refers to an expression that is limited to a specifically defined organ (typically only a single organ).

The term “organ-specific promoter” refers to a promoter that becomes transcribed only in defined organs (typically only a single organ) thus leading to the organ-specific expression.

The term “controllable promoter” refers to a promoter region that is regulated by a transcription factor (enhancer or silencer). “Restricted” is herein understood that in the absence of an appropriate enhancer, no expression is possible, i.e. the promoter therefore shows no basal activity.

The term “partial expression” is understood to mean that the expression rate of a gene is less that that of the corresponding wildtype. Preferably, the rate of expression is maximally 99%, 95%, or 75%, more preferably 50%, and even more preferably the rate is maximally 25% of the expression rate for the wildtype.

“Microinjection” refers to the injection of a substance into a small object, for example a cell or a cell nucleus, using a very fine cannula.

“Pseudo pregnant” refers to a fertile female mouse, whereby after having sexual intercourse with a sterile male mouse, the pregnancy was initiated. According to the present invention, the female mouse receives a manipulated blastocyst into the uterus 2.5 days following such intercourse.

### DETAILED DESCRIPTION OF THE INVENTION

The present invention is carried out using a nonhuman animal model for organ fibrosis, whereby this model comprises a double transgenic nonhuman animal.

In one embodiment, the invention concerns a transgenic, nonhuman animal, which comprises a first recombinant gene that is stably integrated into a genome, said gene coding for a cytokine, whereby the cytokine is expressed in a conditional and organ-specific manner and whereby this expression results in organ fibrosis.

In a preferred embodiment, the expression of the first recombinant gene is controlled through a first controllable promoter, whereby the first controllable promoter preferably comprises a tet-operator sequence.

The first recombinant gene codes for a cytokine. The term cytokine refers to messenger materials that drive the functioning of the immune system, and are therefore of great importance for immune defense. Most cytokines exert their cellular effect by interacting with a receptor on a cell. An overview relating to cytokines, including those cytokines according to the present invention, can be found in Molecular Biology and Biotechnology - A Comprehensive Desk Reference; edited by Robert A. Meyers, VCH Publisher Inc., 1995, pp. 200-204.

An example of a cytokine includes the interleukins, such as the type of interleukins derived from T-cells, B-cells and senescent macrophages.

In a further preferred embodiment of the invention, the first recombinant gene codes for a cytokine, selected from the group of the proinflammatory cytokines, including TGF-γ, IL-1, IL-2 and IL-13.

In an especially preferred embodiment, the nonhuman transgenic animal is further characterized in that it comprises a second recombinant gene that is stably integrated in the genome, which codes for a controllable transactivator protein (tTA).

tTA has the ability to control the activity of the first controllable promoter, and thus the expression of the cytokines. The first promoter comprises, as previously described above, a tet-operator sequence.

The term tTA refers to a “tetracycline-dependent transactivator protein” (Triezenberg S. J. et al., Genes Dev 1988, 2(6):718-29; Gossen M. et al., Science 1995, 268(5218):1766-9), which consists of the E. coli tet-Repressor, that is bound to the transcription activating domain of the Herpes Simplex virus VP16-Proteins.

When the tTA becomes expressed, the resulting protein typically binds with high affinity to the tet-operator sequence that is situated within the first controllable promoter, thus stimulating the expression of the first recombinant gene, i.e. the cytokine. However, with the addition of tetracycline or doxycycline, the affinity of tTA for the tet-operator sequence is lost, thus the tTA protein becomes detached from the tet-operator sequence, resulting in a termination of cytokine transcription.

In a preferred embodiment, the transcription of the cytokines is terminated upon the application of doxycycline (herein “DOX”).

In a particularly preferred embodiment of the invention, tTA is expressed in an organ-specific manner. Such organ-specific expression is ensured as the tTA is expressed under the direction of a second controllable tissue specific promoter.

A preferred embodiment is where the expression of the tTA is limited to one or more hepatocytes. Moreover, a particularly preferred embodiment of the invention comprises a second controllable promoter, whereby the promoter is LAP (i.e. liver enriched activator protein). For liver specific expression, the second controllable promoter is, but not limited to, the transthyretine promoter, the insulin-like-growth-factor promoter, or a promoter derived from various cytochrome P₄₅₀ genes.

Depending on the strength of the TGF-P production and release, the organ fibrosis can be enhanced following brief and repetitive cyclic intervals (i.e. interval treatment) of DOX exposure, namely, the absence of DOX (4-10 day) and presence of DOX (2-5 days). Deviations from this interval treatment model are possible; the intensity of the TGF-β production can be regulated through a constant exposure of a low concentration of DOX (0.2 to 10 μg/ml) in order to achieve a partial expression of the cytokines.

The present invention further comprises a method for making a nonhuman double transgene animal. According to the present invention, two expression vectors are constructed. One of the expression vectors comprises a nucleic acid sequence coding for a cytokine that is under the control of the tet-operator, while the other vector comprises a nucleic acid sequence coding for tTA under the control of an organ-specific promoter.

According to the invention, embryonic stem cells, preferably nonhuman embryonic stem cells, even more preferably embryonic stem cells from a mouse, become separately transfected with the vectors described above. Subsequently, the stem cells are selected; said stem cells contain one of these vectors. Following selection, the embryonic stem cells are microinjected into a blastocyst prior to transplantation into a pseudo pregnant animal.

The present invention provides for two different transgenic lines: one line is transgenic for the cytokine controllable by a tet-operator, while the other line is transgenic for the tTA controllable by an organ-specific promoter. The descendants of both lines are subsequently paired, so as to produce double transgene animals having the ability for both the conditional and organ-specific development of a fibrosis phenotype.

In a preferred embodiment of the inventive method, the expression of the cytokine is inhibited during the embryonic development of the transgenic animals via the application of doxycycline.

For generating the transgenic animals, rodents are preferred, especially mice and rats.

Instead of the controllable transactivator proteins (tTA), the reverse controllable transactivator protein (rtTA) is also suitable. In this case, all method steps remain the same when generating the double transgenic mice, except that there was no fibrogenesis in the absence of DOX and becomes induced following the addition of DOX (therefore the induction of DOX is reversed).

Surprisingly, the method according to the present invention provides a useful animal model, via the use of a double transgene nonhuman animal, for the controllable induction of fibrosis by cytokines without interfering with or otherwise damaging the organism. The presently described double transgene system uses the tTA system to control the organ- and developmental-specific expression of the cytokines.

An additional and surprising finding of the present invention is that in the double transgenic animals, activation and termination of the TGF-β-expression can induce a severe but reversible fibrosis.

The absence or presence of doxycycline governs the activation or termination of TGF-β expression. In animals, in the presence of DOX, no demonstrable TGF-β concentrations can be shown. However, after DOX-removal, the fibrogenesis rapidly appears, as characterized by a significant TGF-β production and a reciprocal increase in the concentration of cytokines in the serum.

Moreover, the animal model of the present invention provides an opportunity to analyze the cause of the fibrosis, depending on the affected organ and the age of the animal. The organ-specific expression depends on the selection of the second, controllable organ-specific promoter that is controlled by the expression of tTA.

The present invention is also suitable for analyzing fibrogenesis in other organs such as, for example, the heart, if suitable second controllable promoters are selected that can be specifically expressed in heart muscle cells. By analogy, this paradigm applies to other organs, including the kidney.

Furthermore, the double transgene animals of the present invention are suitable to show that the fibrogenesis and the other involved cellular reactions are completely reversible. Thus, this animal model is not only suitable for investigating the cause of fibrosis, but additionally for investigating the various mechanisms that contribute to the regeneration of the fibrotic organs. Examples of such mechanisms include, but are not limited to, the apoptosis of myofibroblasts and hepatic star cells, the degradation of extracellular matrix proteins, and the enhanced proliferation of parenchymal cells. Thus, the animal model of the present invention offers the possibility of screening candidate pharmaceuticals or other medical therapies that can support or otherwise participate in the regeneration process following fibrotic lesions.

Moreover, the animal model can be used to examine a cytokine-dependent induction of fibrosis using different types of cytokines. Such a paradigm could distinguish between the various pathologies and associated mechanisms that are involved in the development of fibrosis. The age-dependent induction of fibrosis can also be useful for investigating other types of liver diseases.

### BRIEF DESCRIPTION OF THE DRAWINGS

The invention is further described by the following figures:

FIG. 1 shows the Crossmon trichrome staining of extracellular matrix proteins in liver slices of (A) a double transgenic TGF-β mouse, 8 weeks following the start of an interval treatment through the time the DOX exposure terminated, and (B) a control mouse (without TGF-β expression). The blue staining shows a significant deposit of extracellular matrix proteins in the initiated mouse (A), in the region extending from the periportal areale area into the central areale area.

FIG. 2 shows a steady-state level of the mRNA of procollagen I (alpha 1). The mRNA steady-state levels were determined in control animals (T-LAP2) and double transgenic TGF-β mice after induction with 10 interval cycles (on) and/or after further 6 (“off-6d”) or 21 days (“off-21 d”) upon the application of DOX; the levels are indicated as attog/μg of total RNA. The values represent averages+standard deviation from four different determinations.

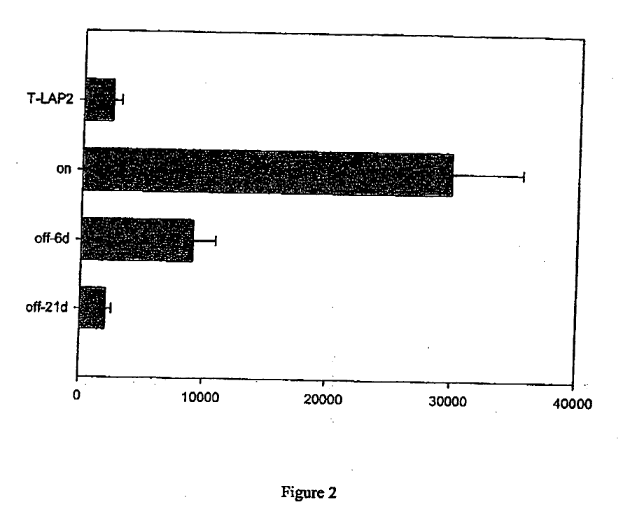

### EXAMPLES

The present invention is further illustrated by way of the following examples, in a non-limiting manner.

### Example 1

Generation of TGF-β1 Transgenic Mice

The construction of the TGF-β1 expression vectors resulted from the cloning of a mutated pig TGF-β1 minimal cDNA (Brunner A. M. et al., J Biol Chem 1989, 264:13660-13664) into a HindIII/Eco RV interface of a tet-cloning vector pBI-5(CVU 89934) (Baron U. et al., Nucleic Acids Res 1997, 25:2723-2729). Prokaryotic sequences were subsequently eliminated using an Ase 1/Xmn I double digest.

Transgenic mouse lines were produced using known standard techniques via microinjection. For the screening of transgenic animals, the DNA was isolated from mice tails using the DNEASY-Kit (Qiagen). PCR and Southern-Blotting were performed using TGF-β1 and luciferase specific primers and corresponding DIG-marked probes.

A further selection criterion that was used related to the differential regulation of the transgenes. For example, primate ear fibroblasts from the transgenic mouse were transfected with the tTA plasmid (PUHD-15.1), and the expression of the luciferase in the presence and the absence of doxycycline hydrochloride in the culture medium was determined.

Founder animals, i.e. animals of the F1 generation having controllable transgenes, were paired with Black-6 mice (C57BL/6NCRLBR, Charles River Laboratories) in order to produce a stable and defined genetic background.

### Example 2

Generation of the Double Transgenic Mice

For the production of the double transgenic mice, the TGF-β1 transgene animals were crossed with representatives from the previously produced transactivator lines TA^{LAP}1/L7 and TA^{LAP}2/L7 (Kistner A. et al., Proc. Soc. Natl. Acad. Sci, USA, 1996; 93:10933-10938), said transactivator cell lines expressing tTA as controlled by the LAP (liver enriched activator protein).

In order to inhibit the expression of TGF-β1 during embryonic development, doxycycline hydrochloride (50 mg/l in a 5% sucrose solution) was added to the drinking water of the pregnant females. The drinking water was changed every two days. Following the birth, the doxycycline remained in the drinking water. The induction of the TGF-β1 expression was initiated at arbitrary time points by removing the doxycycline.

### Example 3

Analysis of the TGF-β Expression

The serum levels of TGF-β1 were determined by means of an ELISA assay (Pharmingen) according to the manufacturer data. Prior to running the ELISA tests, the serum samples were adjusted to an acidic pH. The serum values measured in a time interval of up to five days; the value of TGF-β varied between 250-1200 ng/ml.

### Example 4

Measurement of the Luciferase Activity

The luciferase activity in homogenates derived from liver and kidney (produced with phosphate buffer, pH 7.4) were measured using known techniques (Gaunitz F. et al., Biochem Biophys Res Commun 2001, 284:377-383).

The luciferase activity was measured according to the techniques disclosed by Gaunitz and Papke, Gene Transfer and Expression, In: Methods in Molecular Biology 107 (Phillips IR, Shephard EA, eds.), pp. 361-370, Humana Press, Totowa, N.J., 1997. In the presence of DOX, the luciferase activity was under the detection limit. However, in the absence of DOX, the luciferase activity was measured at levels up to 10,000 rlu/μg Protein (rlu=relative light units).

### Example 5

Induction of Fibrosis

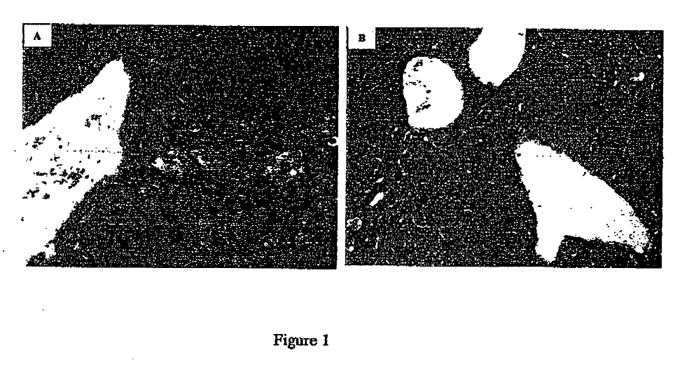

A typical induction scheme was initiated eighty days after the birth of the mice. DOX exposure was terminated for 5 days to a maximum of 10 days, followed by a subsequent exposure for a period of 2 to 3 days. The first indications of fibrosis appeared after 2 weeks. Within 2 months, the fibrosis was readily evident (see FIG. 1). As the course of the interval treatment paradigm continued, a commensurate increase in fibrosis followed, until the onset of liver cirrhosis.

1. A transgenic, nonhuman animal, wherein the transgenic animal comprises a first recombinant gene stably integrated in a genome, said gene codes for a cytokine, whereby the cytokine is expressed in a conditional and organ-specific manner, wherein this expression results in organ fibrosis.

2. A transgenic animal according to claim 1, wherein the expression of the first recombinant gene is controlled by a first controllable promoter.

3. A transgenic animal according to claim 2, wherein the first controllable promoter comprises a tet-operator sequence.

4. A transgenic animal according to claim 1, wherein the cytokine is a proinflammatory cytokine selected from the group consisting of TGF-β, IL-4 and IL-10.

5. A transgenic animal according to claim 1, wherein the cytokine is selectively expressed in hepatocytes.

6. A transgenic animal according to claim 1, wherein the organ fibrosis occurs in liver, heart, kidney, lung or pancreas.

7. A transgenic animal according to claim 1, wherein the animal is a rodent.

8. A transgenic animal according to claim 7, wherein the animal is a mouse or a rat.

9. A transgenic animal according to claim 1, further comprising a second recombinant gene stably integrated into the genome, said gene coding for a controllable transactivator protein (tTA) or a transactivator protein (rTA), wherein the tTA or the rTA controls the first controllable promoter.

10. A transgenic animal according to claim 9, wherein the tTA or the rTA is regulated by doxycycline.

11. A transgenic animal according to claim 1, wherein the intensity of TGF-β production is regulated through a constant exposure to a concentration of DOX for achieving a partial expression of the cytokine, wherein the concentration is from 0.2 μg/ml to 10 μg/ml.

12. A transgenic animal according to claim 1, wherein the organ fibrosis formation can be dependent according to the strength of TGF-β production if brief and repetitive cyclic intervals of the absence of DOX (4-10 days) and the presence of DOX (2-5 days) follow each other.

13. A transgenic animal according to claim 9, wherein the organ-specific expression of the tTA or rTA is controlled by a second controllable promoter.

14. A transgenic animal according to claim 13, wherein the second controllable promoter controls the expression of the tTA or rTA in a hepatocyte.

15. A transgenic animal according to claim 14, wherein the second controllable promoter is LAP.

16. A method for producing a nonhuman transgenic animal according to claim 1, comprising:
a1) constructing an expression vector comprising a cytokine, wherein the cytokine expression is regulated by the tet-promoter;
a2) constructing an expression vector, wherein expressing the tTA or the rTA is regulated by an organ-specific promoter;
b) separately introducing the vector of a1 and the vector of a2 into different nonhuman embryonic stem cells;
c) selecting an embryonic stem cell comprising the vector of a1 or the vector of a2;
d) microinjecting the selected embryonic stem cells into a blastocyst;
e) transplanting the blastocyst into a pseudo pregnant animal;
f1) generating a transgenic animal comprising a transgene having the vector of a1;
f2) generating a transgenic animal comprising a transgene having the vector of a2; and,
g) pairing the transgenic animal of f1 with the transgenic animal of f2 for producing a double transgene animal having the capacity for a conditional and organ-specific development of a fibrosis phenotype.

17. A method according to claim 16, wherein the expression of the cytokine during embryonic development of the transgenic animal is inhibited by doxycycline.

18. Use of a transgenic, nonhuman animal according to claim 1 as a model system for investigating the cause of and a therapy for organ fibrosis.

19. Use according to claim 18, wherein the organ fibrosis is located in a liver, heart, kidney, lung, or pancreas.
